(19) United States
(12) Patent Application Publication
(10) Pub. No.: US 2011/0150743 A1
(43) Pub. Date: Jun. 23, 2011

(54) HYDROCYANIC ACID CONTAINING BIORESOURCE CARBON

(75) Inventor: Jean-Luc Dubois, Millery (FR)

(73) Assignee: Arkema France, Colombes (FR)

(21) Appl. No.: 12/993,374

(22) PCT Filed: May 19, 2009
(86) PCT No.: PCT/FR2009/050926

§ 371 Date: Jan. 21, 2011

(30) Foreign Application Priority Data
May 21, 2008 (FR) 0853286

(51) Int. Cl. C01C 3/02; C01C 3/00; C07C 253/24; C07C 51/305; C01C 3/10

(52) U.S. Cl. 423/376; 423/372; 423/375; 558/316; 558/319; 558/318; 562/538; 562/549; 423/371

(57) ABSTRACT

The invention relates to a hydrocyanic acid containing bioresource carbon, and to a method for producing a raw material mainly containing the same by reacting ammonia with methane or methanol optionally in the presence of air and/or oxygen, characterized in that at least one of the reagents selected from ammonia, methane and methanol is obtained from a biomass. The invention also relates to the uses of the raw material for producing acetone cyanohydrin, adiponitrile, methionine or methionine hydroxyl-analog, and sodium cyanide.

The present invention relates to hydrocyanic acid and has more particularly as subject matter a hydrocyanic acid comprising bioresourced carbon and a process for production of the latter by ammoxidation of methane or methanol.

Hydrocyanic acid HCN has numerous applications as reactant in various synthetic processes or as synthetic intermediate. It is in particular a key reactant in the preparation of acetone cyanohydrin, a synthetic intermediate in the manufacture of organic products, such as methyl methacrylate MMA, or the manufacture of insecticides. Sodium cyanide, a derivative of HCN, also has numerous applications in the chemical industry.

The current industrial production of hydrocyanic acid HON is based mainly on the Andrussow process dating from the 1930s. This process consists in reacting methane or natural gas with ammonia in the presence of air and optionally of oxygen over a catalyst composed of rhodium/platinum gauzes. As the reaction CH₄+NH₃→HCN+3H₂ (1) is endothermic, the addition of air makes it possible, by virtue of the combustion of a portion of the hydrogen produced and of the excess methane, to have a system which is exothermic overall and thus to keep the synthetic reaction going without contributing energy externally.

The reaction, known under the name of ammoxidation, is as follows:

CH₄+NH₃+ 3/2O₂→HCN+3H₂O+heat (2)

The process is based on the reactions (1) and (2).

The kinetics are very fast with a contact time of approximately a few milliseconds or tenths of a millisecond and a velocity of the gases of the order of a few meters per second. The proportion of each reactant is optimized so as to obtain a maximum yield and to avoid the region of inflammability of the reaction mixture.

The reaction generally achieves a yield of 60 to 70%, expressed as number of moles of hydrocyanic acid produced with regard to the number of moles of ammonia introduced, the conversion of the methane being virtually quantitative. The selectivity for hydrocyanic acid, expressed as number of moles of HCN produced with regard to the number of moles of NH₃ which have reacted, is generally from 80 to 90%.

The Degussa process for producing HCN is based on the abovementioned reaction (1), in the absence of oxygen or of air, at a temperature of the order of 1300° C. The reaction is carried out in sintered alumina pipes coated internally with platinum. The bundle of pipes is heated with the gas inside a furnace.

Another process consists in using methanol as replacement for methane in order to produce HCN according to the reaction:

CH₃OH+NH₃+O₂→HCN+3H₂O (3)

This process, described in particular in the 1950-1960s in the patents GB 718 112 and GB 913 836 from Distillers Company, employs a catalyst based on molybdenum oxide at a temperature ranging from 340° C. to 450° C. or a catalyst based on antimony and on tin at a temperature ranging from 350° C. to 600° C. Reference may be made to the article by Walter Sedriks in Process Economics Reviews PEP'76-3, June 1977. This process has formed the subject of various improvements, in particular with regard to the catalytic systems employed; mention may be made, for example, of the systems based on mixed oxides of molybdenum/bismuth/iron supported on silica (U.S. Pat. No. 3,911,089 of Sumitomo, U.S. Pat. No. 4,511,548 of The Standard Oil Company, JP 2002-097017 of Mitsubishi) or the catalysts based on Fe/Sb/O described by Nitto Chemical Industry (EP 340 909, EP 404 529, EP 476 579, Science and Technology in Catalysis, 1998, pages 335-338, Applied Catalysis A: General 194-195, 2000, 497-505) or by Mitsubishi (JP 2002-097015, JP 2002-097016, EP 832 877).

Still other processes exist for producing hydrocyanic acid. Mention may in particular be made of the synthesis according to the Sohio process of acrylonitrile from propylene (reaction A), which generates hydrocyanic acid as byproduct (reaction B):

C₃H₆+NH₃+ 3/2O₂→CH₂═CH—CN+3H₂O (A)

C₃H₆+3NH₃+3O₂→HCN+6H₂O (B)

The HCN yield depends greatly on the operating conditions, on the type of reactor and on the catalyst used. In some cases, it is also possible to add methanol during the ammoxidation of the propylene to increase the production of hydrocyanic acid. This combination is rendered possible in particular by the closeness of the formulations of the catalysts of bismuth molybdate or iron antimonate type and of the operating conditions.

Hydrocyanic acid can also be obtained from the reaction of ammonia with a hydrocarbon, generally propane, in a fluidized bed of coke particles which is heated to a temperature of 1350-1650° C., according to the following reaction (Gulf-Shawinigan process):

C₃H₈+3NH₃→3HCN+7H₂ (C)

Heat is supplied by electrical resistance heaters immersed in the fluidized bed which provides for the transfer of heat. The yields achieved with respect to the ammonia or the propane are of the order of 85%, this process nevertheless requiring a large amount of energy.

The starting materials used in these various processes for the production of hydrocyanic acid (methane, ammonia, propylene, propane) are mainly of fossil or petroleum origin. This is because methane is the main component of natural gas, a fossil fuel composed of a mixture of hydrocarbons naturally present in the gas form in porous rocks. Ammonia is obtained by reaction of atmospheric nitrogen and of hydrogen originating from the steam reforming of the hydrocarbons present in naphtha or in natural gas. Propylene is obtained by steam cracking or catalytic cracking of petroleum fractions. Propane is extracted either from crude oil during refining operations or from natural gas and associated gases in oil deposits.

These various processes thus comprise numerous sources of emissions of CO₂ and contribute to the increase in the greenhouse effect. By way of indication, in the process for the industrial synthesis of ammonia, the cumulative CO₂ emissions are 4300 g/kg of NH₃ and the CO₂ emissions associated with the production of HCN have been evaluated at 4400 g/kg of HCN (Catalysis Today, 99, 2005, 5-14). In addition, these processes employ oil, the deposits of which are rapidly becoming exhausted; its extraction is increasingly difficult (wells of great depth), requiring large-scale and expensive installations which have to withstand high temperatures (400-500° C.). Given the decline in global oil reserves, the source of these starting materials will gradually dry up.

The starting materials resulting from biomass are bioresourced and have a reduced impact on the environment. They do not require all the stages of extraction or of refining, which are very expensive in terms of energy, of oil products. The CO₂ production is reduced, so that they contribute less to climate warming and meet some concerns of sustainable development.

It thus appears necessary to have available processes for the synthesis of hydrocyanic acid which are not dependent on a starting material of fossil origin but instead use starting materials of renewable origin, that is to say comprising bioresourced carbon.

The problem which the present invention intends to solve is that of conceiving of hydrocyanic acid comprising bioresourced carbon; this is obtained from biomass. The term “biomass” is understood to mean living starting material, of vegetable or animal origin, produced naturally. The vegetable material is characterized in that the plant, for its growth, has consumed carbon dioxide gas while producing oxygen. The animals, for their growth, have for their part consumed this vegetable starting material and have thus taken in the carbon derived from atmospheric CO₂. Biomass is regarded as the energy source having the greatest potential (heat, electricity, hydrogen) since it is regarded as neutral with regard to the formation of CO₂.

Due to the extreme sensitivity of the catalysts to poisoning by certain impurities, the starting materials employed in the processes for the production of HCN have to have a satisfactory quality and a satisfactory purity. In particular, in the process of the ammoxidation of methane, use is made of methane with a purity of greater than 91% which comprises the minimum of higher hydrocarbons (ethane and in particular propane) and which is devoid of sulfur. The fluctuation in the quality of the natural gas generally presents problems for the catalytic reaction for the ammoxidation of methane. The ammonia is filtered and evaporated and preferably comprises neither oils nor iron.

The aim of the present invention is thus to provide a process for the production of hydrocyanic acid, based on the use of starting materials comprising bioresourced carbon, of uniform quality, which does not require preliminary stages of purification of the starting materials, which is easy to employ and which is easily adapted to the devices existing in industry for the manufacture of hydrocyanic acid.

The hydrocyanic acid according to the invention comprises bioresourced carbon; more specifically, it comprises ¹⁴C.

This is because, unlike materials resulting from fossil materials, starting materials of renewable origin comprise ¹⁴C in the same proportions as atmospheric CO₂. All the samples of carbon drawn from living organisms (animals or plants) are in fact a mixture of 3 isotopes: ¹²C (representing approximately 98.892%), ¹³C (approximately 1.108%) and ¹⁴C (traces: 1.2×10⁻¹⁰%). The ¹⁴C/¹²C ratio of living tissue is identical to that of the atmosphere. In the environment, ¹⁴C exists in two predominant forms: in the mineral form, that is to say in the form of carbon dioxide gas (CO₂), and in the organic form, that is to say in the form of carbon incorporated in organic molecules.

In a living organism, the ¹⁴C/¹²C ratio is kept constant by the metabolism as the carbon is continually exchanged with the environment. As the proportion of ¹⁴C in the atmosphere is constant, it is the same in the organism while it is living, since it absorbs this ¹⁴C as it absorbs the ¹²C. The mean ¹⁴C/¹²C ratio is equal to 1.2×10⁻¹² for a material of renewable origin, while a fossil starting material has a zero ratio.

¹²C is stable, that is to say that the number of ¹²C atoms in a given sample is constant over time. ¹⁴C for its part is radioactive and its concentration decreases over time; its half life is 5730 years.

In view of the half life of ¹⁴C, the ¹⁴C content is substantially constant from the extraction of the renewable starting materials up to the manufacture of the “biomaterials” resulting from these starting materials and even up to the end of their use.

The ¹⁴C content of a “biomaterial” can be deduced from measurements carried out, for example, according to the following techniques:
By liquid scintillation spectrometry: this method consists in counting the “Beta” particles resulting from the disintegration of the ¹⁴C. The Beta radiation resulting from a sample of known weight (known number of carbon atoms) is measured for a certain time. This “radioactivity” is proportional to the number of ¹⁴C atoms, which can thus be determined. The ¹⁴C present in the sample emits β radiation which, on contact with the liquid scintillant (scintillator), gives rise to photons. These photons have different energies (of between 0 and 156 keV) and form what is referred to as a ¹⁴C spectrum. According to two alternative forms of this method, the analysis relates either to the CO₂ produced beforehand by combustion of the carbon-comprising sample in an appropriate absorbent solution or to the benzene after preliminary conversion of the carbon-comprising sample to benzene.
By mass spectrometry: the sample is reduced to graphite or to CO₂ gas and analyzed in a mass spectrometer. This technique uses an accelerator and a mass spectrometer to separate the ¹⁴C ions from the ¹²C ions and thus to determine the ratio of the two isotopes.

These methods for measuring the ¹⁴C content of the materials are clearly described in the standards ASTM D 6866 (in particular D6866-06) and in the standards ASTM D 7026 (in particular 7026-04). These methods compare the data measured on the sample analyzed with the data of a reference sample comprising 100% of bioresourced carbon (for which ¹⁴C/¹²C has the value 1.2×10⁻¹²), to give a relative percentage of bioresourced carbon in the sample. The ¹⁴C/¹²C ratio of the sample can subsequently be deduced therefrom.

The measurement method preferably used is the mass spectrometry described in the standard ASTM D 6866-06 (accelerator mass spectroscopy).

A subject matter of the present invention is thus a hydrocyanic acid, characterized in that it comprises a content by weight of ¹⁴C such that the ¹⁴C/¹²C ratio is between 0.2×10⁻¹² and 1.2×10⁻¹² according to the standard ASTM D 6866; preferably, the ¹⁴C/¹²C ratio is between 0.6×10⁻¹² and 1.2×10⁻¹². In a preferred embodiment, the hydrocyanic acid of the invention is such that the ¹⁴C/¹²C ratio is equal to 1.2×10⁻¹², that is to say that it comprises 100% of bioresourced carbon.

Another subject matter of the present invention is a process for the synthesis of a starting material comprising mainly hydrocyanic acid by reaction of ammonia with methane or methanol, optionally in the presence of air and/or of oxygen, characterized in that one at least of the reactants chosen from ammonia, methane and methanol is obtained from biomass.

The term “starting material comprising mainly hydrocyanic acid” means that the process results in the production of hydrocyanic acid optionally comprising impurities related to the nature of the reactants employed or generated during the process, it being possible for this hydrocyanic acid subsequently to be used as starting material in organic syntheses.

### Increase in Value of Biomass as Ammonia

In accordance with a first embodiment, the ammonia was obtained from hydrogen resulting from a syngas (composed essentially of carbon monoxide and hydrogen) resulting from the gasification of biomass.

Gasification is a thermochemical process which makes it possible to produce a gas rich in hydrogen from biomass and a gaseous reactant, such as air, oxygen or steam. The conversion takes place at high temperature (800-1000° C.) and generally at atmospheric pressure or slight excess pressure. The concentration of oxygen (in the air or the water) is not sufficient during the gasification to result in complete oxidation. Thus, large amounts of CO and H₂ are produced according to the following reactions:

C+H₂O→CO+H₂

C+CO₂→2CO

At the same time, other gaseous products (CH₄, heavier hydrocarbons, CO₂ but also NH₃, sulfur-comprising or chlorine-comprising gases, NOₓ) and solid products (tars, charcoal and dust) can be formed in a small amount according to the conditions employed.

Use may be made, as biomass, of any material of animal or vegetable origin. The materials of animal origin are, as nonlimiting examples, fish oils and fats, such as cod liver oil, whale oil, sperm whale oil, dolphin oil, seal oil, sardine oil, herring oil or shark oil, oils and fats of bovines, porcines, caprines, equids and poultry, such as tallow, lard, milk fat, pig fat, chicken, cow, pig or horse fats, and others. The materials of vegetable origin are, for example, vegetable oils, cereal straw fodder, such as wheat straw fodder or corn straw fodder; cereal residues, such as corn residues; cereal flours, such as wheat flour; cereals, such as wheat, barley, sorghum or corn; wood or wood waste and scraps; grains; sugar cane or sugar cane residues; pea tendrils and stems; beets or molasses, such as beet molasses; potatoes, potato haulms or potato residues; starch; mixtures of cellulose, hemicellulose and lignin; or black liquor from the papermaking industry.

The gas composition of the mixture produced depends on numerous factors, such as the composition of the reaction mixture (presence or absence of nitrogen in a large amount), the water content, the design of the gasification reactor (fixed or fluidized bed reactor) or the temperature of the reaction. Gasification reactions are highly endothermic. The simplest route for providing the heat necessary consists in using air as gasification agent and in thus partially incinerating the biomass. Use may advantageously be made of steam as oxidizing agent with the aim of maximizing the production of hydrogen.

Conventional technologies for the gasification of biomass are essentially of two types: fixed bed processes, in which the solid fuel introduced into the upper part descends by gravity into the reactor and reacts on contact with the oxidizing agent, generally air or oxygen, and fluidized bed processes, in which the biomass, reduced in size (a few tens of millimeters) and dried beforehand, is introduced, solid or liquid, into a bed of sand, which improves the heat transfer and the material transfer. Other technologies can be used, in particular the “Chemrec” technology, for example described in the document FR 2 544 758, suitable for the gasification of papermaking pulp spent liquor. This technology is based on combustion between 1000° C. and 1300° C. in a reaction zone in which external heat energy is provided independently of the combustion.

The hydrogen, after conversion by steam of the carbon monoxide product of the syngas, is purified before being introduced into a catalytic reactor for the synthesis of ammonia at high pressure (100 to 250 bar).

According to a preferred embodiment of the invention, the hydrogen used to prepare the ammonia originates from the recovery of spent liquor from the manufacture of cellulose pulps. Reference may be made to the documents FR 2 544 758, EP 666 831 or U.S. Pat. No. 7,294,225 of Chemrec, which describe in particular the gasification of spent liquors from the manufacture of cellulose.

### Increase in Value of Biomass as Methane

In accordance with a second embodiment, the methane was obtained from biogas. Biogas is the gas produced by the fermentation of animal and/or vegetable organic matter in the absence of oxygen. This fermentation, also known as methanization, takes place naturally or spontaneously in landfill sites containing organic waste but can be carried out in digesters, in order to treat, for example, sewage sludge, industrial or agricultural organic waste, pig manure or household refuse. Preferably, use is made of biomass containing animal manure which acts as nitrogenous input necessary for the growth of the microorganisms providing the fermentation of the biomass to give methane. The biogas is composed essentially of methane and carbon dioxide gas; the carbon dioxide gas is subsequently removed by washing the biogas using a basic aqueous sodium hydroxide, potassium hydroxide or amine solution or also with water under pressure or by absorption in a solvent, such as methanol. It is possible to obtain, according to this route, pure methane of uniform quality. Reference may be made to the various methanization technologies of the state of the art, in the paper Review of Current Status of Anaerobic Digestion Technology for Treatment of Municipal Solid Waste, November 1998, RISE-AT, and to the various existing biological processes for the treatment of waste water, such as, for example, the Laran® process of Linde.

### Increase in Value of Biomass as Methanol

In accordance with a third embodiment, the methanol was obtained from the pyrolysis of wood.

In accordance with a fourth embodiment, the methanol was obtained by fermentation of crops of plants, such as wheat, sugar cane or beet, giving fermentable products.

In accordance with a fifth embodiment, the methanol was obtained by gasification of any material of animal or vegetable origin, resulting in a syngas composed essentially of carbon monoxide and hydrogen which is reacted with water. The materials of animal or vegetable origin are those described above as starting materials in the production of ammonia by increase in value of biomass.

It would not be departing from the scope of the invention to use methane resulting from biogas to produce the syngas.

According to a preferred embodiment of the invention, the syngas for preparing the methanol originates from the recovery of spent liquor from the manufacture of cellulose pulps. Reference may be made to the documents EP 666 831 and U.S. Pat. No. 7,294,225 of Chemrec, which describe in particular the gasification of spent liquors from the manufacture of cellulose and the production of methanol, and to pages 92-105 of the work Procédés de pétrochimie—Caractéristiques techniques et économiques—Tome 1—Editions Technip—le gaz de synthése et ses dérivés [Petrochemical processes—Technical and Economic Characteristics—Volume 1—Published by Technip—Syngas and its derivatives], which relates to the production of methanol from syngas.

### Manufacture of the Starting Material Comprising Mainly Hydrocyanic Acid

In accordance with a first embodiment of the process according to the invention, ammonia is reacted with methane in the presence of air and optionally of oxygen over a catalyst composed of rhodium/platinum gauzes at a temperature ranging from 1050 to 1150° C. Generally, the CH₄/NH₃ molar ratio ranges from 1.0 to 1.2 and the total (CH₄+NH₃)/O₂ molar ratio ranges from 1.6 to 1.9; the pressure is generally from 1 to 2 bar.

In accordance with a second embodiment of the process according to the invention, ammonia is reacted with methanol at a temperature ranging from 350° C. to 600° C. in the presence of a catalyst, for example a catalyst based on molybdenum/bismuth/iron supported on silica or a catalyst based on antimony and iron.

Use may in particular be made, for this reaction, of the operating conditions and the catalysts described in the abovementioned documents U.S. Pat. No. 3,911,089, U.S. Pat. No. 4,511,548, JP 2002-097017, EP 340 909, EP 404 529, EP 476 579, JP 2002-097015, JP 2002-097016 and EP 832 877.

The process according to the invention can additionally comprise one or more purification stages.

The starting material obtained according to the process of the invention is different from the product which can be obtained according to conventional processes for the manufacture of hydrocyanic acid starting from starting materials of fossil origin; it comprises the conventional byproducts of these processes, such as the unreacted reactants, described in Ullmann's Encyclopedia of Industrial Chemistry, Vth Edn. (1987), Vol. A8, pages 161-163, but can comprise impurities related to the nature of the reactants employed or generated during the process. It can be used, optionally after a purification stage, in processes employing hydrocyanic acid as starting material.

Thus, the invention also relates to the use of the starting material comprising mainly hydrocyanic acid according to the invention in the manufacture of acetone cyanohydrin. The reaction between this starting material and acetone to give acetone cyanohydrin is generally carried out in the liquid phase at a temperature of the order of 25° C. to 40° C., at atmospheric pressure, with an HCN/acetone molar ratio of the order of 0.7 to 1.1.

The acetone cyanohydrin is an intermediate compound in the production of methyl methacrylate (MMA) according to two possible routes: a first route consists in forming, by reaction of sulfuric acid with acetone cyanohydrin, α-hydroxyisobutyramide monosulfate, which is converted to methacrylamide sulfuric acid. The latter is subsequently hydrolyzed and esterified with methanol to form methyl methacrylate.

A second route consists in directly reacting methanol with acetone cyanohydrin and in then carrying out a dehydration reaction to result in methyl methacrylate.

Reference may be made to the article Techniques de l'Ingénieur, traité Génie des Procédés [Techniques of the Engineer, Process Engineering Treatise], J 6-400-1 to 6, which describes the conditions for the industrial implementation of the process for the production of methyl methacrylate according to the acetone cyanohydrin route.

The acetone cyanohydrin is used more generally in the manufacture of organic products and of insecticides.

The starting material obtained from the process according to the invention is also used to produce adiponitrile by reaction with butadiene according to the reaction:

CH₂═CH—CH═CH₂+2HCN→NC—(CH₂)₄—CN

The adiponitrile, after hydrogenation, results in hexamethylenediamine, which is an intermediate compound in the production of polyamide 6,6 (Nylon®) by polycondensation of hexamethylenediamine adipate.

Reference may be made to the article Techniques de l'Ingénieur, traité Génie des Procédés, J 6-515-1 to 7, which describes the synthesis of polyamide 6,6 according to this route.

Advantageously, the starting material obtained from the process according to the invention is used in a process for the synthesis of methionine or methionine hydroxy analogue. The chemical processes made use of industrially are based essentially on the same main starting materials and the same key intermediates, namely:
acrolein CH₂═CH—CHO and methyl mercaptan CH₃SH (MSH), resulting by reaction in methylmercapto-propionaldehyde CH₃—S—CH₂—CH₂—CHO (MMP), also denoted by 3-(methylthio)propanal or by methylthiopropionaldehyde (MTPA),
hydrocyanic acid (HCN) or sodium cyanide (NaCN), which, after reaction with MMP, finally results in methionine or methionine hydroxy analogue.

Reference may be made to the article Techniques de l'Ingénieur, traité Génie des Procédés, J 6-410-1 to 9, which describes the conditions for the industrial implementation of the processes for the synthesis of methionine involving methylmercaptopropionaldehyde as intermediate and using hydrocyanic acid as reactant.

Advantageously, the starting material obtained from the process according to the invention is also used to produce sodium cyanide by neutralization with sodium hydroxide according to the reaction:

HCN+NaOH→NaCN+H₂O

Sodium cyanide has numerous applications, in particular for the extraction of precious metals, electroplating or the synthesis of chemical compounds.

1. A hydrocyanic acid, characterized in that it comprises a content by weight of ¹⁴C such that the ¹⁴C/¹²C ratio is between 0.2×10⁻¹² and 1.2×10⁻¹² according to the standard ASTM D 6866.

2. A process for the synthesis of hydrocyanic acid by reaction of ammonia with methane or methanol, optionally in the presence of air and/or of oxygen, characterized in that at least one of said ammonia, methane or methanol is obtained from biomass.

3. The process as claimed in claim 2, characterized in that the ammonia is obtained from hydrogen resulting from a syngas (CO/H₂) resulting from the gasification of biomass.

4. The process as claimed in claim 2, characterized in that the methane is obtained from biogas (CH₄/CO₂) produced by the fermentation of animal or vegetable organic matter in the absence of oxygen, the CO₂ being removed by washing the biogas using a basic aqueous sodium hydroxide, potassium hydroxide or amine solution or with water under pressure or by absorption in a solvent.

5. The process as claimed in claim 2, characterized in that the methanol is obtained from the pyrolysis of wood.

6. The process as claimed in claim 2, characterized in that the methanol is obtained by fermentation of crops of plants.

7. The process as claimed in claim 2, characterized in that the methanol is obtained by gasification of any material of animal or vegetable origin, resulting in a syngas composed essentially of carbon monoxide and hydrogen which is reacted with water.

8. The process as claimed in claim 7, characterized in that the syngas results from spent liquor from the manufacture of cellulose pulps.

9. The process as claimed in claim 2, characterized in that ammonia is reacted with methane in the presence of air and optionally of oxygen over a catalyst composed of rhodium/platinum gauzes at a temperature ranging from approximately 1050-1150° C.

10. The process as claimed in claim 2, characterized in that ammonia is reacted with methanol at a temperature ranging from 350° C. to 600° C. in the presence of a catalyst.

11-14. (canceled)

15. A method of producing a hydrocyanic acid selected from the group consisting of acetone cyanohydrin, adiponitrile, methionine, methionine hydroxy analogue and sodium cyanide comprising reacting ammonia with methane or methanol, optionally in the presence of air and/or of oxygen, wherein at least one of said ammonia, methane or methanol is obtained from biomass.
